(19) United States
(12) Patent Application Publication
(10) Pub. No.: US 2025/0387026 A1
(43) Pub. Date: Dec. 25, 2025

(54) SYSTEM AND METHOD FOR INTERVENTIONAL PLANNING FOR THE TREATMENT OF BRAIN DISORDERS

(71) Applicant: TURING MEDICAL TECHNOLOGIES INC., St. Louis, MO (US)

(72) Inventors: Nico DOSENBACH, St. Louis, MO (US); Damien FAIR, St. Louis, MO (US)

(21) Appl. No.: 19/111,261

(22) PCT Filed: Sep. 13, 2023
(86) PCT No.: PCT/US2023/074054

§ 371 Date: Mar. 12, 2025

Related U.S. Application Data
(60) Provisional application No. 63/375,454, filed on Sep. 13, 2022.

(51) Int. Cl. A61B 5/00; A61B 5/055; G01R 33/56; G01R 33/563; G06T 7/00; G06T 7/215

(52) U.S. Cl. CPC A61B 5/0042; A61B 5/055; G01R 33/5608; G01R 33/56341; G06T 7/0016; G06T 7/215; G06T 2207/30016; G06T 2207/30168

(57) ABSTRACT

A computer-implemented method for brain mapping and target identification for interventional planning using magnetic resonance imaging (MRI) includes receiving, by a computing system that includes at least one processor in communication with at least one memory system and that is in communication to receive data acquired using an MRI system, MR data from the MRI system. The method further includes analyzing the received MR data to monitor and identify motion in real-time, determining a set of useable MR data from the acquired MR data based on the identified motion, generating a map of the subject's brain based on the set of useable MR data and identifying a target location in the subcallosal cingulate (SCC) region of the subject's brain based on the map of the subject's brain. The target location can be a point of convergence of multiple fiber bundles passing through the SCC region. The method can further include generating a report indicating the target location.

### CROSS-REFERENCE TO RELATED APPLICATIONS

This application is based on, claims priority to, and incorporates herein by reference in its entirety U.S. Ser. No. 63/375,454 filed Sep. 13, 2022, and entitled “System and Method for Computer Aided Diagnosis (CAD) for Surgical Planning for the Treatment of Mental Disorders.”

### BACKGROUND

Psychiatric disorders are a common cause of severe and long-term disability and socioeconomic burden. In some patients, treatment modalities of drug therapy and psycho-therapy do not produce sufficient therapeutic effects or induce intolerable side effects. For these patients, neuromodulation has been suggested as a potential treatment modality. Neuromodulation is one of the fastest-growing areas of medicine, and is the process of inhibition, stimulation, modification, regulation or therapeutic alteration of activity, electrically or chemically, in the central, peripheral or autonomic nervous systems. Neuromodulation incudes deep brain stimulation, vagal nerve stimulation, and transcranial magnetic and electrical stimulation. Neuromodulation aims to treat chronic neurological or psychiatric diseases by surgically targeting deep brain nuclei and pathways involved in the mediation of the symptoms in order to stimulate, inhibit, or otherwise modify/modulate pathological activity.

Neural structures such as cortical and/or subcortical structures are targeted using deep brain stimulation (DBS) for treatment of neurological and psychiatric disorders, including essential tremor, Parkinson disease, dystonia, Tourette syndrome, obsessive compulsive disorder, and treatment-resistant depression. Yet, specific target structures have variable success rates. DBS of ventral intermediate nucleus of the thalamus for treatment of essential tremor results in over 80% tremor reduction in all patients, while stimulation of the globus pallidus for treatment of dystonia results in only 30-50% symptom improvement across all patients and >75% improvement in only 33% of patients.

Body motion, such as head motion, represents the greatest obstacle to collecting quality brain Magnetic Resonance Imaging (MRI) data in humans. Head motion distorts structural (T1-weighted, T2-weighted, etc.), functional MRI (task driven [fMRI] and resting state functional connectivity [rs-fcMRI]), and diffusion MRI (e.g., diffusion tensor imaging (DTI)) data. Even sub-millimeter head movements (e.g., micro-movements) may systematically alter structural, functional, and diffusion MRI data in some cases. Hence, much effort has been devoted toward developing post-acquisition methods for the removal of head motion distortions from MRI data.

Head movement from one MRI data frame (or slice) to the next, rather than absolute movement away from the reference frame, is thought to induce the most significant MRI signal distortions. Motion-related distortions are strongly correlated with measures of framewise displacement (FD, which represents the sum of the absolute head movements in all six rigid body directions from frame to frame), zipper artifacts or outlier slices, neighboring DWI correlation on the raw data, as well as DVARS (the RMS of the derivatives of the differentiated timecourses of every voxel of an MRI image). Thus, measures such as FD and DVARS that capture the global effects of movement of the subject during MRI data acquisition have been used to assess data quality in various post-hoc methods. For example, post-hoc frame censoring which removes all MRI data frames with FD values above a certain threshold (for example, excluding data frames with FD values>0.2 mm) has become a commonly used method for improving functional MRI data quality.

Though necessary for reducing artifacts, frame censoring comes at a steep price. For example, frame censoring can exclude 50% or more of rs-fcMRI data collected from a cohort depending on one's specific parameters and the quality of the underlying data. Because the accuracy of MRI measures improves as the number of frames increases, a minimum number of data frames nay be required to obtain reliable data. If the number of fames remaining after censoring is too small, investigators may lose all data from a participant. In order to avoid this loss, investigators typically collect additional “buffer” data, an expensive practice that, by itself, does not guarantee sufficient high-quality MRI data for a given participant. The “overscanning” required to remove motion-distorted data while maintaining sample sizes adequate to achieve a desired data quality has drastically increased the cost and duration of brain MRIs.

Recently developed structural MRI sequences with prospective motion correction use a similar approach to reduce the deleterious effects of head motion. These MRI sequences pair each structural data acquisition with a fast, low resolution, snapshot of the whole brain (echo-planar image=EPI), which is then used as a marker or navigator for head motion. These motion-correcting structural sequences calculate relative motion between successive navigator images and used this information to mark the linked structural data frames for exclusion and reacquisition. In this manner, structural data frames are “censored” thereby increasing the duration and cost of structural MRIs.

For structural, functional, and diffusion MRI, access to real-time information about in-scanner head movement while scanning could greatly reduce the costs of MRI by eliminating the need for overscanning. The assessment of head movement obtained from real-time motion monitoring would allow scanner operators to continue each scan until the desired number of low-movement data frames have been acquired without need for excess buffer scans. Existing approaches to real-time motion monitoring measure proxies for FD using expensive cameras and lasers. Unfortunately, such proxies of head movement are poorly correlated with FD because these proxies typically cannot distinguish movements of the face and scalp from brain movements.

### SUMMARY OF THE DISCLOSURE

In accordance with an embodiment, a computer-implemented method for brain mapping and target identification for interventional planning using magnetic resonance imaging (MRI) includes receiving, by a computing system that includes at least one processor in communication with at least one memory system and that is in communication to receive data acquired using an MRI system, MR data from the MRI system. The method further includes analyzing the received MR data to monitor and identify motion in real-time, determining a set of useable MR data from the acquired MR data based on the identified motion, generating a map of the subject's brain based on the set of useable MR data and identifying a target location in the subcallosal cingulate (SCC) region of the subject's brain based on the map of the subject's brain. The target location can be a point of convergence of multiple fiber bundles passing through the SCC region. The method can further include generating a report indicating the target location.

In some embodiments, the multiple fiber bundles passing through the SCC region includes cingulum bundle (CM), forceps minor (FM), frontal striatal fibers (F-ST), and uncinate fasciculus (UF). In some embodiments, the method further includes displaying the report on a display. In some embodiments, the received MR data is diffusion MR data. In some embodiments, the received diffusion MR data is acquired using one of diffusion tensor imaging (DTI) or diffusion weighted imaging (DWI). In some embodiments, the received diffusion MR data is acquired for a fist number of diffusion directions. In some embodiments, the method further includes determining additional diffusion directions different from the first number of diffusion directions based on the identified motion and set of useable MR data. In some embodiments, the method further includes receiving, by the computer system, additional MR data acquired for the additional diffusion directions from the MRI system.

In accordance with another embodiment, a system for brain mapping and target identification for interventional planning using magnetic resonance imaging (MRI) includes a computing device and a display. The computing device include a processor programmed to receive MR data acquired using an MRI system, analyze the received MR data to monitor and identify motion in real-time, determine a set of useable MR data from the acquired MR data based on the identified motion, generate a map of the subject's brain based on the set of useable MR data and identify a target location in the subcallosal cingulate (SCC) region of the subject's brain based on the map of the subject's brain. The target location can be a point of convergence of multiple fiber bundles passing through the SCC region. The processor is further programmed to generate a report indicating the target location. The display is coupled to the computing device and is configured to display the report.

In some embodiments, the multiple fiber bundles passing through the SCC region includes cingulum bundle (CM), forceps minor (FM), frontal striatal fibers (F-ST), and uncinate fasciculus (UF). In some embodiments, the received MR data is diffusion MR data. In some embodiments, the received diffusion MR data is acquired using one of diffusion tensor imaging (DTI) or diffusion weighted imaging (DWI). In some embodiments, the received diffusion MR data is acquired for a fist number of diffusion directions. In some embodiments, the processor is further programmed to determine additional diffusion directions different from the first number of diffusion directions based on the identified motion and set of useable MR data. In some embodiments, the processor is further programmed to receive additional MR data acquired for the additional diffusion directions from the MRI system.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 illustrates an example method for performing mapping of and identifying target locations in a subject's brain for interventional planning in accordance with an embodiment;

FIG. 2 shows an example display of a target location in a subcallosal cingulate (SCC) region of the brain in accordance with an embodiment;

FIG. 3 is a flow chart illustrating the operations for aligning magnetic resonance imaging (MRI) data from a MRI scan to a selected frame in the MRI scan in accordance with an embodiment;

FIG. 4 is a flow chart illustrating a method for the generation of a sensory feedback display to the operator of the MRI system and/or the patient within the MRI system during data acquisition in accordance with an embodiment;

FIG. 5 is a schematic diagram of an example system for performing magnetic resonance imaging in accordance with an embodiment;

FIG. 6A is a block diagram of an example of a system for brain mapping and target identification for interventional planning for treatment of brain disorders in accordance with an embodiment; and

FIG. 6B is a block diagram of components that can implement the system for brain mapping and target identification for interventional planning for treatment of brain disorders of FIG. 6A in accordance with an embodiment.

### DETAILED DESCRIPTION

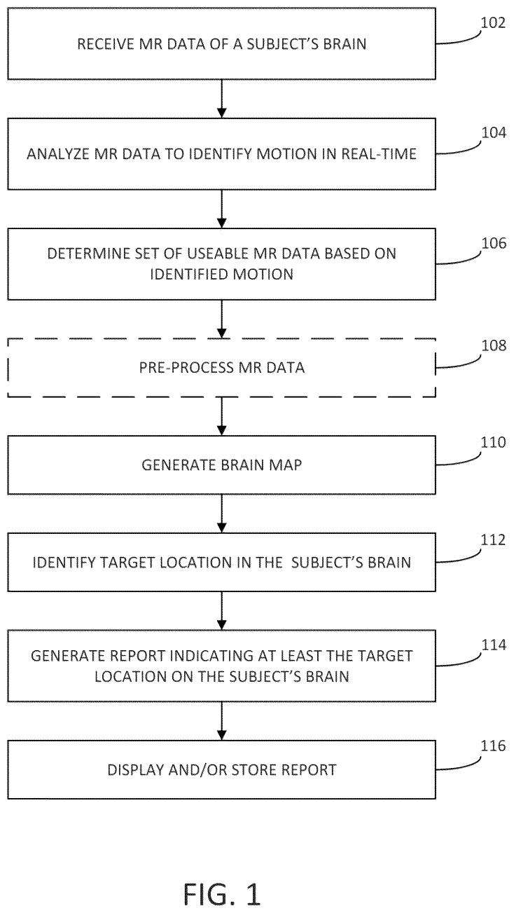

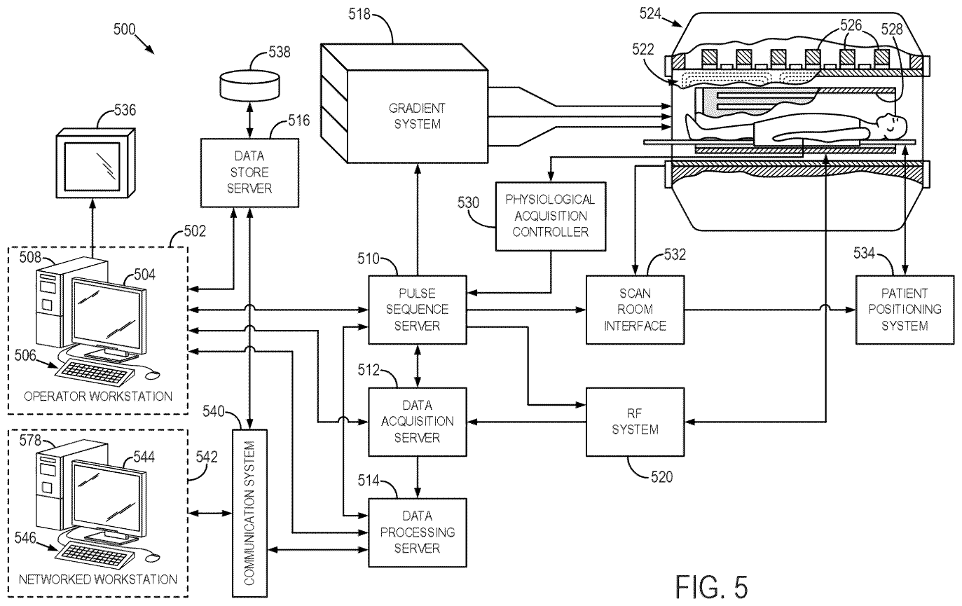

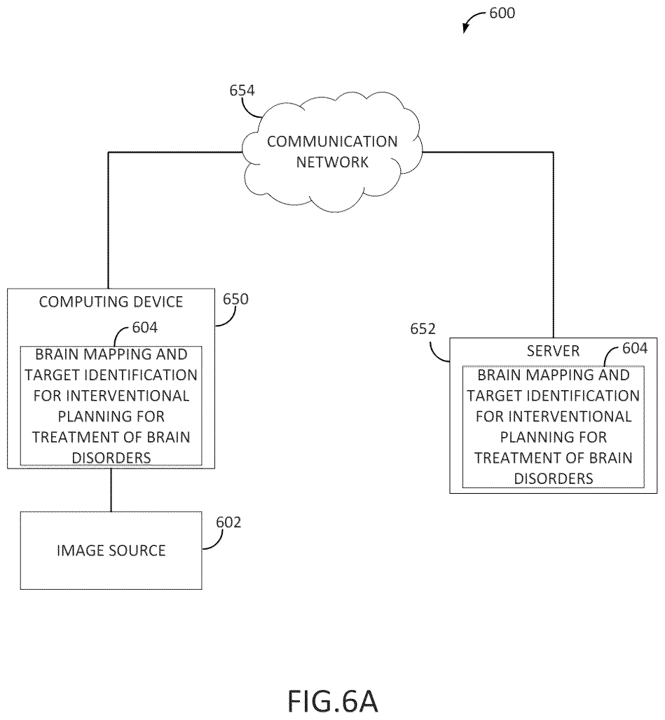

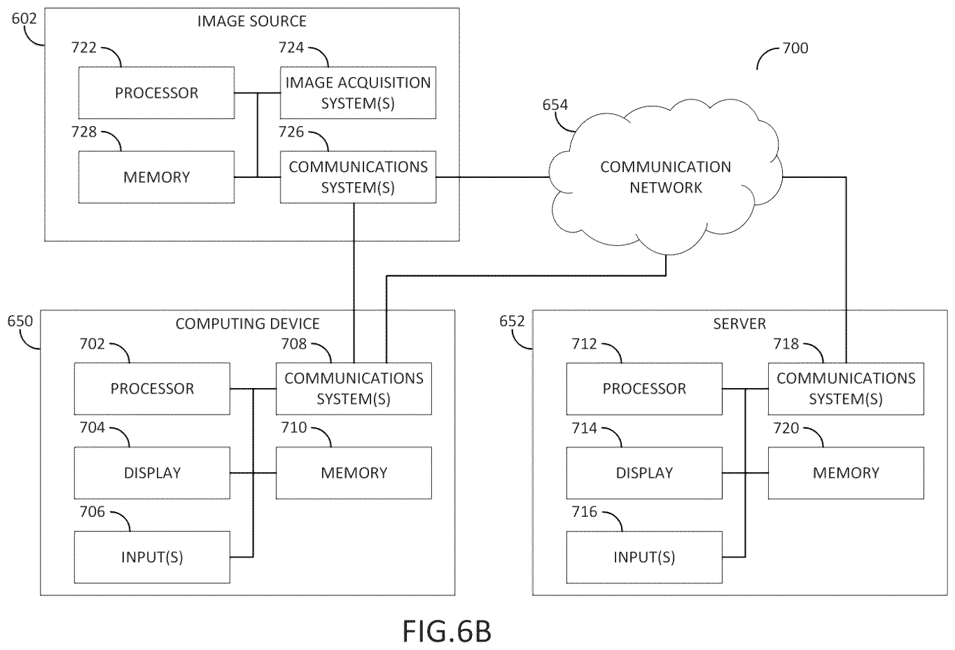

FIG. 1 illustrates an example method for performing mapping of and identifying target locations in a subject's brain in accordance with an embodiment. Although the blocks of the process of FIG. 1 are illustrated in a particular order, in some embodiments, one or more blocks may be executed in a different order than illustrated in FIG. 1, or may be bypassed. The method includes, at block 102, receiving magnetic resonance (MR) data of a brain of a subject such as, for example, structural (T1-weighted, T2-weighted) MRI data, functional MRI data, and diffusion MRI data. In some embodiments, the MR data of the brain of the subject may be acquired using, for example, diffusion imaging techniques (e.g., DTI or DWI) or functional magnetic resonance imaging (fMRI) techniques. The fMRI data of the brain of the subject may include task driven (fMRI) data, resting-state fMRI (rs-fMRI)) data, or combination thereof. A subject may be a human, an animal, a phantom, or the like. In some embodiments, the MR data may be acquired by and received from an MRI system (e.g., MRI system 500 shown in FIG. 5) in real time. In some embodiments, the MR data may be retrieved from data storage of an imaging system (e.g., disc storage 538 of MRI system 500 shown in FIG. 5), or data storage of other computer systems (e.g., memory 710 of computer device 650, or memory 720 of server 652 shown in FIG. 6B).

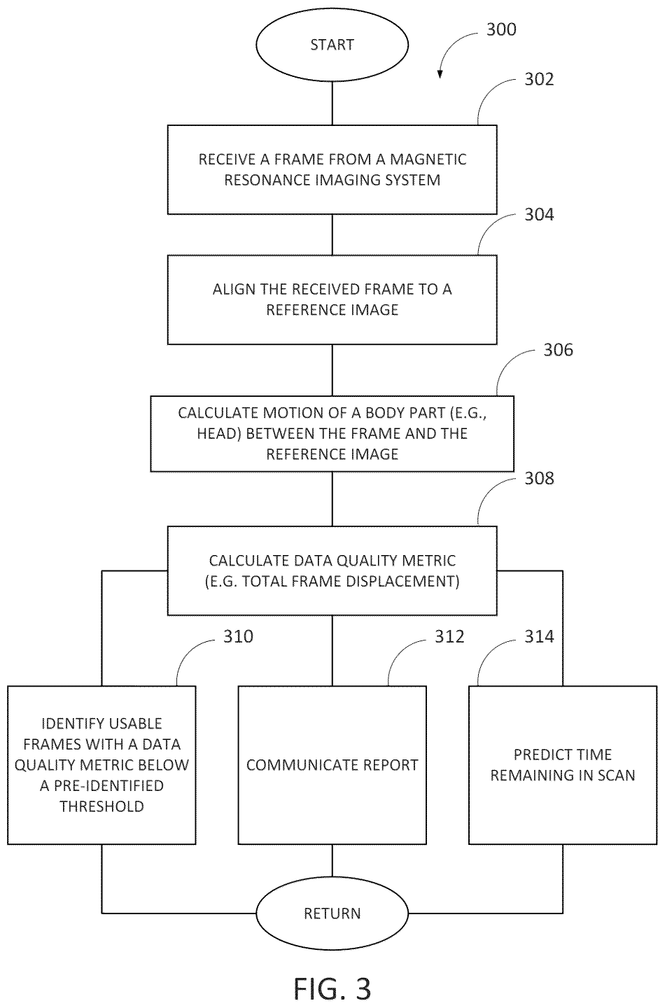

At block 104, the MR data may be analyzed to identify motion in real time. At block 106, a set of useable MR data (e.g., non-motion corrupted MR data) may be determined based on the motion identified at block 104. In some embodiments, blocks 104 and 106 may be performed as part of the acquisition of the MR data at block 102. For example, blocks 102, 104 and 106 may include using systems, devices and methods for real-time monitoring and prediction of motion of a body part of a patient including, but not limited to, head motion during MRI scanning. In some embodiments, acquisition of the MR data may include using Framewise Integrated Real-time MRI Monitoring (FIRMM) systems, devices and methods as described further below with respect to FIG. 3, to address motion-induced artifacts. Real-time monitoring and prediction of degraded data quality may include, but is not limited to, patient motion (e.g., head motion) during scanning.

For the purposes of this disclosure and accompanying claims, the term “real time” or related terms are used to refer to and defined a real-time performance of a system, which is understood as performance that is subject to operational deadlines from a given event to a system's response to that event. For example, a real-time extraction of data and/or displaying of such data based on empirically-acquired signals may be one triggered and/or executed simultaneously with and without interruption of a signal-data acquisition (e.g., pulse sequence) or imaging procedure.

At block 108, in some embodiments, the MR data or images acquired at block 102 or the useable MR data determined at block 106 may be optionally preprocessed for the mapping process. For example, in some embodiments, high resolution T1 images may be preprocessed by performing skull stripping, image registration and normalization to a template, and tissue segmentation, for example, estimating a brain mask for gray matter (GM), white matter (WM), and cerebrospinal fluid (CBF). In some embodiments, diffusion weighted imaging (DWI) data (or images) may be preprocessed by performing skull stripping, simultaneous eddy current and distortion correction (e.g., by registering the diffusion weighted (DW) images to the B0 images with an affine transformation), image registration to B0 image of first acquisition, image registration to T1 image, and local tensor (DTI) fitting. In some embodiments, a transformational matrix between diffusion and T1 concatenated with a previously calculated non-linear normalization field between T1 and the template may be used to create diffusion to the template transformation field.

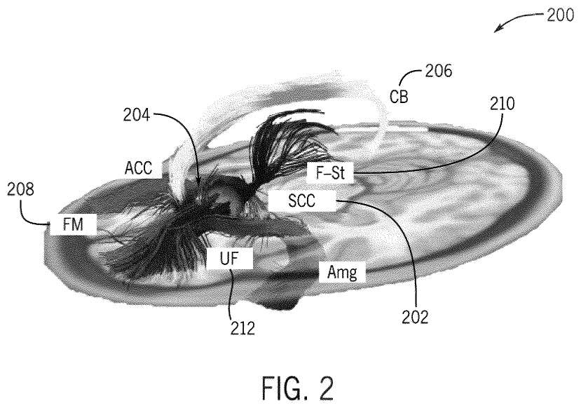

At block 110, the method can include calculating and generating a map (e.g., a functional connectivity map of the brain, tractography, etc.) based at least on the acquired MR data. The method can then include, at block 112, identifying a target location in the brain of the subject to be targeted by, for example, neuromodulation, based on the calculated brain map. In some embodiments, the identification of the target location may be identified using methods that enable personalized patient-specific targeting. In some embodiments, the identified region may be in the subcallosal cingulate (SCC) region of the brain. FIG. 2 shows an example display 200 of a target location in a subcallosal cingulate (SCC) region of the brain in accordance with an embodiment. Advantageously, in some embodiments, the target location 204 may be a point of convergence of multiple fiber bundles passing through the SCC region 202. For example, as shown in FIG. 2, the target location 204 can be a point of convergence of four converging bundles including the cingulum bundle (CB) 206, forceps minor (FM) 208, frontal striatal fibers (F-St) 210, and uncinate fasciculus (UF) 212. The target location 204 may be defined to impact the four bundles (e.g., an implanted neuromodulator at the target location would impact the four bundles). In some embodiments, the target location 204 may be identified automatically.

Returning to FIG. 1, at block 112, in some embodiments, the target location may be, for example, the ventral capsule/ventral striatum (VC/VS), the nucleus accumbens (NAcc), the habenula (LHb), the inferior thalamic bundle (ITP)), medial forebrain bundle (MFB), the bed nucleus of the stria terminalia (BNST), the dentate nucleus, the centromedian nucleus of the thalamus, the ventrointermediate (VIM) nucleus of the thalamus, or the red nucleus. In some embodiments using diffusion MR data, the mapping and target location identification operations 110 and 112, respectively, can include a diffusion tensor imaging (DTI) tracking technique. For a DTI fiber tracking technique, typically using more directions (diffusion gradients) for the diffusion enables better resolution of individual fibers. However, the more directions that are used, the longer the scan may take. In some embodiments, an operator may select a first number of diffusion directions for a scan. Then, based on the feedback (or results) from the real-time monitoring of motion and determining a set of useable MR data (e.g., blocks 104 and 106), the operator may determine whether additional directions are needed in an additional scan. For example, an operator may first select to do three directions and then based on the motion information from block 104, the operator may determine that a second scan with three more directions should be performed. In another example, the operator may first select to do three directions and then based on the motion information from block 104, the operator may determine that another scan with additional different directions is not needed. In some embodiments, the additional different diffusion directions may be determined automatically based on the feedback (or results) from the real-time monitoring of motion and determining a set of useable MR data.

Irrespective of the particular region of the brain being studied, a report may be generated at block 114 that at least indicates the target location. In some embodiments, the report may include a display that includes a visual indicator identifying the target location on, for example, an image or map of the subject's brain. In some embodiments, the report may include a connectome of the subject's brain. At block 116, the generated report may be displayed on a display (for example, displays 504, 536, 544 of MRI system 500 shown in FIG. 5, display 704 of computing device 650 shown in FIG. 6B or display 714 of server 652 shown in FIG. 6B). As one non-limiting example, the target location may be a target location for an intervention such as neuromodulation. The method may further facilitate, for example, surgical planning for implantation of a neuromodulation device and even the ultimate performance of neuromodulation directed at the identified target location. In some embodiments, the reports, images and maps created using the systems and methods disclosed herein may be used to perform interventional planning such as, for example, surgical (e.g., tumor resection) or therapeutic (treatment) planning.

In some embodiments, the systems and methods disclosed herein may be used for interventional planning (e.g., surgical and treatment planning) for treatments of particular brain disorders and in particular structures of the brain. As used herein, the term brain disorders is used to refer to neurological and psychological disorders. For example, various target locations (e.g., the SCC region, the ventral capsule/ventral striatum (VC/VS), the nucleus accumbens (NAcc), the habenula (LHb), the inferior thalamic bundle (ITP)), medial forebrain bundle (MFB), or the bed nucleus of the stria terminalia (BNST)) may be used for the treatment of depression, various target locations (e.g., the dentate nucleus) may be used for motor stroke recover, various target locations (e.g., the centromedian nucleus of the thalamus, the red nucleus) may be used for the treatment of epilepsy, various target locations (e.g., the centromedian nucleus) may be used for the treatment of Tourette's syndrome, various target locations (e.g., the centromedian nucleus of the thalamus, the red nucleus) may be used for the treatment of disorders of consciousness (coma), various target locations (e.g., ventrointermediate (VIM) nucleus of the thalamus, the red nucleus) may be used for the treatment of essential tremor, and various target locations (e.g., the ventrointermediate (VIM) nucleus of the thalamus) may be used for the treatment of tremor predominant Parkinson's.

Deep brain stimulation (DBS) is a form of neuromodulation in clinical use. DBS is a procedure in which a neurostimulator is surgically implanted into the brain for the purpose of treating brain disorders, such as Parkinson's disease, dystonia, essential tremor, obsessive compulsive disorder, epilepsy, depression, etc. In some embodiments, the identified target location may be used to guide planning for DBS lead placement. For example, a physician may review a suggested target location, e.g., on a display, and determine whether to select the target location for lead placement. In some embodiments, a target location in the subcallosal cingulate may be used for deep brain stimulation for depression. Other treatment approaches include, for example, transcranial magnetic stimulation (TMS), transcranial direct current stimulation (tDCS) and focused ultrasound. In some embodiments, the identified target location may be used to guide planning for neuromodulation by providing targets for TMS, tDCS, and focused ultrasound.

As mentioned above, in some embodiments, at block 102, the MR data of a subject's brain may advantageously be acquired using Framewise Integrated Real-time MRI Monitoring (FIRMM) systems, devices and methods for real-time monitoring and prediction of motion of a body part of a patient including, but not limited to, head motion during MRI scanning. An example of FIRMM systems and methods is described in U.S. Pat. No. 11,181,599, issued Nov. 23, 2021, and herein incorporated by reference in its entirety. The FIRMM computer implemented method can simultaneously improve MRI data quality and reduce costs associated with MRI data acquisition. In some embodiments, the FIRMM method may be implemented in the form of a software suite that calculates and displays data quality metrics and/or summary motion statistics in real time during an MRI data acquisition. The FIRMM methods and systems are typically described herein in the context of functional MRI data acquisition, but in various embodiments the FIRMM methods and systems disclosed herein are suitable for real-time monitoring of head and body motion during other structural or anatomical MRI sequences, including but not limited to those that utilize motion navigation. Advantageously, the FIRMM systems and methods can provide real-time feedback to both the scanner operator and the subject undergoing the scan. More specifically, in some embodiments, the FIRMM systems and methods can provide sensory feedback to a subject during the scan based on the data quality metrics and summary motion statistics calculated in real time, thereby enabling the subject to monitor and adjust their movements accordingly (e.g., remain still) in response to the provided feedback. In some embodiments, the FIRM systems and methods can provide stimulus conditions, such as viewing a fixation crosshair or a movie clip, to simultaneously engage the subject while also providing real-time feedback to the subject.

In some embodiments, the FIRMM system and method can enable a scanner operator to continue each scan until the desired number of low-movement data frames have been acquired by, as non-limiting examples, (i) predicting the number of usable data frames that will be available at the end of the scan; (ii) predicting the amount of time a given subject will likely have to be scanned until the preset time-to-criterion (minutes of low-movement FD data) has been acquired; and (iii) enabling for the selection and deselection of specific individual scans for inclusion in the actual and predicted amount of low-movement data.

Real-time information about head motion can be used to reduce head motion in multiple different ways including, but not limited to: 1) by influencing the behavior of MRI scanner operators and 2) by influencing MRI scanning subject behavior. Scanner operators may be alerted about any sudden or unusual changes in head movement and can be enabled to interrupt such scans to investigate if the subject has started moving more because they have grown uncomfortable and whether a bathroom break, blanket, repositioning, or other intervention could make them feel more comfortable. In some embodiments, the FIRMM methods can further include options for feeding information about head motion back to the subject, post-scan and/or in real time. In some embodiments, the FIRMM methods can allow scanner operators to find the sweet spot that provides the required amount of low-movement data at the lowest cost. A scan could be stopped, the subject could be further instructed or reminded on ways to try remaining still, and the scan could be re-acquired, and the like, to address motion.

FIG. 3 illustrates an example FIRMM method 300 for processing a set of MRI frames to align the frames to a reference image in a set to compensate for a subjects' movement. Although the blocks of the process of FIG. 3 are illustrated in a particular order, in some embodiments, one or more blocks may be executed in a different order than illustrated in FIG. 3, or may be bypassed. The method 300, at block 302, can include receiving MR data from a magnetic resonance imaging system in the form of an MRI frame or image. The MRI frame may be received by a computing device from a magnetic resonance imaging system via a network or from a storage medium coupled to or in communication with the computing device.

At block 304, the method 300 can also include aligning the frame to a reference frame or image. In some embodiments, the reference image may be a single frame selected from the frames collected from the MRI scan including, but not limited to, the first frame, a navigator frame, or any other suitable frame selected from a plurality of frames collected during an MRI scan. In some embodiments, the reference image may be an image retrieved from an anatomical atlas. In some embodiments, a composite or combination of two or more frames collected during an MRI scan including, but not limited to, a mean of two or more frames. In some embodiments, each current frame may be aligned to a previous frame collected immediately prior, which has been aligned iteratively with the reference image collected for a given MRI scan.

Each frame may be aligned to the reference image through a series of rigid body transforms, Tᵢ, where i indexes the spatial registration of frame i to a reference frame 1, starting with the second frame. Each transform is calculated by minimizing the registration error to an absolute minimum or below a selected cutoff or otherwise reaching a stop condition relative to a registration error, expressed as:

$${ε}_{i} = 〈 {( {sI}_{i} ( T ( \vec{x} ) ) - {I}_{1} ( \vec{x} ) )}^{2} 〉 \qquad Eqn . ( 1 )$$

where I(\vec{x}) is the image intensity at locus \vec{x} and s is a scalar factor that compensates for fluctuations in mean signal intensity, spatially averaged over the whole brain (angle brackets). In certain aspects, the frames may be realigned using 4dfp cross_realign3d_4dfp algorithm (see Smyser, C. D. et al. 2010, Cerebral cortex 20, 2852-2862, (2010)) which is specifically incorporated herein by reference). Alternative alignment algorithms can also be utilized to align the frame.

In some embodiments, each transform may be represented by a combination of rotations and displacements as described by:

$${T}_{i} = [ \begin{matrix}{R}_{i} & {d}_{i} \\ 0 & 1\end{matrix} ] \qquad Eqn . 2$$

where Rᵢ represents the 3×3 matrix of rotations including the three elementary rotations at each of the three axes and di represents the 3×1 column vector of displacements. Rᵢ may include the three elementary rotations at each of the three axes as expressed by: Rᵢ=R_{iα}R_{iβ}R_{iγ}, where

$${R}_{i α} = [ \begin{matrix}1 & 1 & 0 \\ 0 & cos ( {α}_{i} ) & - sin ( {α}_{i} , ) \\ 0 & sin ( {α}_{i} ) & cos ( {α}_{i} )\end{matrix} ] \qquad Eqn . 3$$

$${R}_{i β} = [ \begin{matrix}cos ( {β}_{i} ) & 0 & sin {β}_{i} \\ 0 & 1 & 0 \\ - sin {β}_{i} & 0 & cos {β}_{i}\end{matrix} ] \qquad Eqn . 4$$

$${R}_{i γ} = [ \begin{matrix}cos ( {γ}_{i} ) & - sin ( {γ}_{i} ) & 0 \\ sin ( {γ}_{i} ) & cos ( {γ}_{i} ) & 0 \\ 0 & 0 & 1\end{matrix} ] \qquad Eqn . 5$$

At block 306, the method 300 can also include calculating the relative motion of a body part (e.g., head) between the frame and the preceding frame. The relative motion of a body part (e.g., head motion) may be calculated from multiple frame alignment parameters including, but not limited to, x, y, z, θₓ, θ_{y}, and θ_{z}, where x, y, z are translations in the three coordinate axis and θₓ, θ_{y}, and θ_{z} are rotations about those axis.

At block 308, the method 300 can also include calculating a data quality metric (e.g., the total frame displacement) using the multiple frame alignment parameters. In some embodiments, the total frame displacement may be determined using multiple displacement vectors of head motion. By way of non-limiting example, total frame displacement may be calculated by adding the absolute displacement of the body part (e.g., head) in six directions, thereby treating the body part as a rigid body. In this non-limiting example, the head motion of the i^{th} frame may be converted to a scalar quantity using the formula:

$${Displacement}_{i} = \vert Δ {d}_{i x} \vert + \vert Δ {d}_{i y} \vert + \vert Δ {d}_{i z} \vert + \vert {Δα}_{i} \vert + \vert {Δβ}_{i} \vert + \vert {Δγ}_{i} \vert \qquad Eqn . 6$$

where Δdᵢₓ=d₍ᵢ₋₁₎ₓ−dᵢₓ; Δd_{iy}=d_{(i-1)y}−d_{iy}; Δd_{iz}=d_{(i-1)z}−d_{iz}; and so forth.

Rotational displacements |Δαᵢ|, |Δβᵢ|, |Δγᵢ| may be converted from degrees to millimeters by computing displacement on the surface of a 3D volume representative of the body part being imaged. By way of non-limiting example, if the head is imaged, the 3D volume selected to calculate displacement may be a sphere (e.g., a sphere of radius 50 mm, which is approximately the mean distance from the cerebral cortex to the center of the head for a healthy young adult). Since each data frame is realigned to the reference image, frame displacement (FD) may be calculated by subtracting Displacementᵢ₋₁ (for the previous frame) from Displacementᵢ (for the current frame).

In some embodiments, the method 300 may farther include excluding frames with a cutoff above a pre-identified threshold of total frame displacement at block 310. In some embodiments, the method may predict whether there will be at least n number of usable frames at the end of an MRI scan. In some embodiments, predicting the number of usable frames includes applying a linear model (y=mx+b), where y is the predicted number of good frames at the end of the scan, x is the consecutive frame count, and m and b are estimated for each subject in real time. In some embodiments, each frame may be labeled as usable if the relative object displacement of that frame is less than a given threshold (e.g., in mm), using the object's position on a previous frame as a reference. One non-limiting example of a cutoff threshold for usable data frames is 0.2, however, in some embodiments, the scan operator can edit a setting file associated with a FIRMM software suite to select a different threshold as desired.

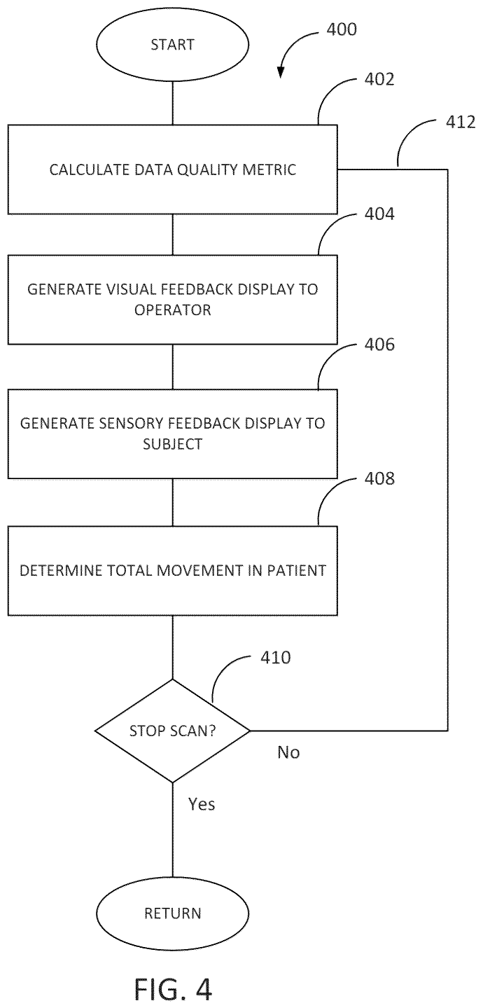

Upon completion, the method 300 can return to the start for each subsequent frame in the MRI scan. A display of the data quality metric and other motion monitoring information may be performed at block 312. In some embodiments, as discussed below with respect to FIG. 4, the motion monitoring information may be provided to the operator and/or the subject undergoing the MRI scan. In some embodiments, a visual display of parameters for the scan may be displayed to an operator. In some embodiments, FD may be provided to the operator in real time, such that each time a new frame/scan/volume is acquired, a new data-point is added to a FD-vs-frame #graph. In some embodiments, at the end of each scan a summary of counts for that scan may be displayed in a list that tabulates the summary head motion data for each scan separately and/or for the sum of all the data acquired thus far in the active scanning session. A prediction of the time remaining in a scan (e.g., until a preset time-to-criterion (minutes of low-movement FD data)) may be performed at block 314. For example, a graph of the actual amount of time (e.g., in min and s or percentages) elapsed to scan “high-quality” frames toward a preset criterion amount of time may be provided. Such information may be provided in the form of a visual display, an auditory signal, or any other known means of providing information without limitation.

As mentioned above, in some embodiments the FIRMM method can generate a sensory feedback display to be communicated to the operator and/or the subject undergoing the MRI scan via a suitable feedback device. Any sensory feedback display may be provided by the FIRMM method via the feedback device including, but not limited to, a visual feedback display, an auditory feedback display, or any other suitable sensory feedback display to any known sensory modality.

FIG. 4 is a flow chart illustrating a method for providing a sensory feedback to the operator of the MRI system and/or the patient within the MRI scanner of the MRI system during data acquisition in accordance with an embodiment. Although the blocks of the process of FIG. 4 are illustrated in a particular order, in some embodiments, one or more blocks may be executed in a different order than illustrated in FIG. 4, or may be bypassed. At block 402, the method 400 can include calculating a data quality metric based on one or more components of movement determined for the patient in the MRI device during scanning as described above with respect to FIG. 3. Any data quality metric may be calculated at block 402 without limitation as described herein including, but not limited to, any one or more of the displacement components as described above with respect to FIG. 3, other data quality metrics including DVARS (i.e., the RMS of the derivatives of the time courses of every voxel of an MRI image), or any combination thereof.

At block 404, the method 400 may further include generating a visual display in real time to an operator of the MRI system based on at least a position of the data quality metric calculated at block 402. Non-limiting examples of suitable visual feedback displays include at least a portion of a GUI, a light bar, a video, an image, and the like. In some embodiments, the visual feedback display for the operator of the MRI system may include visual elements including, but not limited to, one or more graphs displaying the data quality metrics for all frames received in the scan, tables of summary statistics regarding the quality of the current and previous scans, graphical or tabular elements communicating the cumulative number of useable frames obtained in the current scan, tabular or graphical elements communicating the amount of time remaining in the current scan and/or the predicted amount of time remaining in the current scan to obtain a predetermined number of useable scans, and any combination thereof. In some embodiments, the elements of the visual feedback display may be updated a preselected rate up to a real-time rate of updating each display as each relevant quantity is calculated, the elements of the visual feedback display may be updated in response to a request from the operator of the MRI system, and the elements of the visual feedback display may dynamically update in response to at least one of a plurality of factors including, but not limited to, significant increases in the monitored motion of the subject between frames, cumulative motion, or any other suitable criteria.

At block 406, the method 400 may further include generating a sensory feedback display for the patient in the scanner during acquisition of MRI data. The sensory feedback display generated at block 406 may be updated at a wide variety of refresh rates ranging from s single update at the end of scanning to continuously updating in real time, based on at least one of a plurality of factors including, but not limited to the patients age and condition.

At block 408, the method 400 may further include determining the total movement of the patient between the previous frame and the current frame in response to the sensory feedback display generated at block 406. In some embodiments, the method 400 further includes evaluating at least one a plurality of factors to determine whether the current MRI scan should be terminated at block 410. In some embodiments, the scan may be terminated in accordance with at least one of a plurality of termination criteria including, but not limited to, one or more movements of an unacceptably high magnitude, and unacceptably high number of relatively low magnitude movements, a determination that a suitable number of useable frames were obtained, a prediction that a suitable number useable frames cannot be obtained in the time remaining in the scan, a prediction that a suitable number of useable frames cannot be obtained within a reasonable cumulative scan time, and any combination thereof. If it is determined at block 410 to continue the scan, the method 400 may communicate at least one feedback signal 412 to be used in part to calculate the data quality metric at 402 to start another iteration of the method 400 for a subsequent frame.

As mentioned above, in some embodiments, the mapping and target identification operations 104 and 106 discussed above with respect to FIG. 1 can include a DTI fiber tracking technique. For a DTI fiber tracking technique, typically using more directions (diffusion gradients) for the diffusion enables better resolution of individual fibers. However, the more directions that are used, the longer the scan may take. In some embodiments using a FIRMM method for data acquisition, an operator may select a first number of diffusion directions for a scan. Then, based on the feedback (or results) from the real-time monitoring and prediction of degraded data quality, the operator may determine whether additional directions (diffusion gradients) are needed in an additional scan. For example, an operator may first select to do three directions and then based on the motion information from the FIRMM method, the operator may determine that a second scan with three more directions should be performed. In another example, an operator may first select to do three directions and then based on the motion information from the FIRMM method, the operator may determine that another scan with additional different directions is not needed. In some embodiments, the additional different diffusion directions may be determined automatically based on the feedback (or results) from the real-time monitoring of motion and determining a set of useable MR data.

In some embodiments, the methods described herein may be implemented by a system that includes an MRI system and one or more processors or computing devices. In various aspects, one or more operations described herein may be implemented by one or more processors having physical circuitry programmed to perform the operations. In various other aspects, one or more steps of the method may automatically be performed by one or more processors or computing devices. In various additional aspects, the various acts illustrated in FIGS. 1, 3 and 4 may be performed in the illustrated sequence, in other sequences, in parallel, or in some cases, may be omitted.

In some aspects, the above described methods and processes may be implemented using a computing system, including one or more computers. The methods and processes described herein may be implemented as a computer application, computer service, computer API, computer library, and/or other computer program product.

Referring to FIG. 5, an example of an MRI system 500 that can implement the methods described here is illustrated. The MRI system 500 includes an operator workstation 502 that may include a display 504, one or more input devices 506 (e.g., a keyboard, a mouse), and a processor 508. The processor 508 may include a commercially available programmable machine running a commercially available operating system. The operator workstation 502 provides an operator interface that facilitates entering scan parameters into the MRI system 500. The operator workstation 502 may be coupled to different servers, including, for example, a pulse sequence server 510, a data acquisition server 512, a data processing server 514, and a data store server 516. The operator workstation 502 and the servers 510, 512, 514, and 516 may be connected via a communication system 540, which may include wired or wireless network connections.

The pulse sequence server 510 functions in response to instructions provided by the operator workstation 502 to operate a gradient system 518 and a radiofrequency (“RF”) system 520. Gradient waveforms for performing a prescribed scan are produced and applied to the gradient system 518, which then excites gradient coils in an assembly 522 to produce the magnetic field gradients Gₓ, G_{y}, and G_{z} that are used for spatially encoding magnetic resonance signals. The gradient coil assembly 522 forms part of a magnet assembly 524 that includes a polarizing magnet 526 and a whole-body RF coil 528.

RF waveforms are applied by the RF system 520 to the RF coil 528, or a separate local coil to perform the prescribed magnetic resonance pulse sequence. Responsive magnetic resonance signals detected by the RF coil 528, or a separate local coil, are received by the RF system 520. The responsive magnetic resonance signals may be amplified, demodulated, filtered, and digitized under direction of commands produced by the pulse sequence server 510. The RF system 520 includes an RF transmitter for producing a wide variety of RF pulses used in MRI pulse sequences. The RF transmitter is responsive to the prescribed scan and direction from the pulse sequence server 510 to produce RF pulses of the desired frequency, phase, and pulse amplitude waveform. The generated RF pulses may be applied to the whole-body RF coil 528 or to one or more local coils or coil arrays.

The RF system 520 also includes one or more RF receiver channels. An RF receiver channel includes an RF preamplifier that amplifies the magnetic resonance signal received by the coil 528 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} \qquad Eqn . 7$$

and the phase of the received magnetic resonance signal may also be determined according to the following relationship:

$$φ = {tan}^{- 1} ( \frac{Q}{I} ) \qquad Eqn . 8$$

The pulse sequence server 510 may receive patient data from a physiological acquisition controller 530. By way of example, the physiological acquisition controller 530 may receive signals from a number of different sensors connected to the patient, including electrocardiograph (“ECG”) signals from electrodes, or respiratory signals from a respiratory bellows or other respiratory monitoring devices. These signals may be used by the pulse sequence server 510 to synchronize, or “gate,” the performance of the scan with the subject's heart beat or respiration.

The pulse sequence server 510 may also connect to a scan room interface circuit 532 that receives signals from various sensors associated with the condition of the patient and the magnet system. Through the scan room interface circuit 532, a patient positioning system 534 can receive commands to move the patient to desired positions during the scan.

The digitized magnetic resonance signal samples produced by the RF system 520 are received by the data acquisition server 512. The data acquisition server 512 operates in response to instructions downloaded from the operator workstation 502 to receive the real-time magnetic resonance data and provide buffer storage, so that data is not lost by data overrun. In some scans, the data acquisition server 512 passes the acquired magnetic resonance data to the data processor server 514. In scans that require information derived from acquired magnetic resonance data to control the further performance of the scan, the data acquisition server 512 may be programmed to produce such information and convey it to the pulse sequence server 510. For example, during pre-scans, magnetic resonance data may be acquired and used to calibrate the pulse sequence performed by the pulse sequence server 510. As another example, navigator signals may be acquired and used to adjust the operating parameters of the RF system 520 or the gradient system 518, or to control the view order in which k-space is sampled. In still another example, the data acquisition server 512 may also process magnetic resonance signals used to detect the arrival of a contrast agent in a magnetic resonance angiography (“MRA”) scan. For example, the data acquisition server 512 may acquire magnetic resonance data and processes it in real-time to produce information that is used to control the scan.

The data processing server 514 receives magnetic resonance data from the data acquisition server 512 and processes the magnetic resonance data in accordance with instructions provided by the operator workstation 502. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 514 are conveyed back to the operator workstation 502 for storage. Real-time images may be stored in a data base memory cache, from which they may be output to operator display 502 or a display 536. Batch mode images or selected real time images may be stored in a host database on disc storage 538. When such images have been reconstructed and transferred to storage, the data processing server 514 may notify the data store server 516 on the operator workstation 502. The operator workstation 502 may be used by an operator to archive the images, produce films, or send the images via a network to other facilities.

The MRI system 500 may also include one or more networked workstations 542. For example, a networked workstation 542 may include a display 544, one or more input devices 546 (e.g., a keyboard, a mouse), and a processor 548. The networked workstation 542 may be located within the same facility as the operator workstation 502, or in a different facility, such as a different healthcare institution or clinic.

The networked workstation 542 may gain remote access to the data processing server 514 or data store server 516 via the communication system 540. Accordingly, multiple networked workstations 542 may have access to the data processing server 514 and the data store server 516. In this manner, magnetic resonance data, reconstructed images, or other data may be exchanged between the data processing server 514 or the data store server 516 and the networked workstations 542, such that the data or images may be remotely processed by a networked workstation 542.

Referring now to FIG. 6A, an example of a system 600 for in accordance with some embodiments of the systems and methods described in the present disclosure is shown. As shown in FIG. 6A, a computing device 650 can receive one or more types of data (e.g., MR data) from image source 602, which may be an MRI source. In some embodiments, computing device 650 can execute at least a portion of a system 604 for brain mapping and target identification for interventional planning for treatment of brain disorders that can include correction for motion in data received from the image source 602.

Additionally or alternatively, in some embodiments, the computing device 650 can communicate information about data received from the image source 602 to a server 652 over a communication network 654, which can execute at least a portion of the system 604 for brain mapping and target identification for interventional planning for treatment of brain disorders. In such embodiments, the server 652 can return information to the computing device 650 (and/or any other suitable computing device) indicative of an output of the system 604.

In some embodiments, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. The computing device 650 and/or server 652 can also reconstruct images from the data.

In some embodiments, image source 602 can be any suitable source of image data (e.g., measurement data, images reconstructed from measurement data), such as a magnetic resonance imaging system (e.g., MRI system 500 shown in FIG. 5), another computing device (e.g., a server storing image data), and so on. In some embodiments, image source 602 can be local to computing device 650. For example, image source 602 can be incorporated with computing device 650 (e.g., computing device 650 can be configured as part of a device for capturing, scanning, and/or storing images). As another example, image source 602 can be connected to computing device 650 by a cable, a direct wireless link, and so on. Additionally or alternatively, in some embodiments, image source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 654 can be a local area network, a wide area network, a public network (e.g., the Internet), a private or semi-private network (e.g., a corporate or university intranet), any other suitable type of network, or any suitable combination of networks. Communications links can each be any suitable communications link or combination of communications links, such as wired links, fiber optic links, Wi-Fi links, Bluetooth links, cellular links, and so on.

Referring now to FIG. 6B, an example of hardware 700 that can be used to implement image source 602, computing device 650, and server 652 in accordance with some embodiments of the systems and methods described in the present disclosure is shown. As shown in FIG. 6B, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (“CPU”), a graphics processing unit (“GPU”), and so on. In some embodiments, display 704 can include any suitable display devices, such as a computer monitor, a touchscreen, a television, and so on. In some embodiments, inputs 706 can include any suitable input devices and/or sensors that can be used to receive user input, such as a keyboard, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include RAM, ROM, EEPROM, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., images, user interfaces, graphics, tables), receive content from server 652, transmit information to server 652, and so on.

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EEPROM, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some embodiments, image source 602 can include a processor 722, one or more image acquisition systems 724, one or more communications systems 726, and/or memory 728. In some embodiments, processor 722 can be any suitable hardware processor or combination of processors, such as a CPU, a GPU, and so on. In some embodiments, the one or more image acquisition systems 724 are generally configured to acquire data, images, or both, and can include an MRI imaging system. Additionally or alternatively, in some embodiments, one or more image acquisition systems 724 can include any suitable hardware, firmware, and/or software for coupling to and/or controlling operations of an MRI system. In some embodiments, one or more portions of the one or more image acquisition systems 724 can be removable and/or replaceable.

Note that, although not shown, image source 602 can include any suitable inputs and/or outputs. For example, image source 602 can include input devices and/or sensors that can be used to receive user input, such as a keyboard, a mouse, a touchscreen, a microphone, a trackpad, a trackball, and so on. As another example, image source 602 can include any suitable display devices, such as a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more image acquisition systems 724, and/or receive data from the one or more image acquisition systems 724; to images from data; present content (e.g., images, a user interface) using a display; communicate with one or more computing devices 650; and so on. Memory 728 can include any suitable volatile memory, non-volatile memory, storage, or any suitable combination thereof. For example, memory 728 can include RAM, ROM, EEPROM, one or more flash drives, one or more hard disks, one or more solid state drives, one or more optical drives, and so on. In some embodiments, memory 728 can have encoded thereon, or otherwise stored therein, a program for controlling operation of image source 602. In such embodiments, processor 722 can execute at least a portion of the program to generate images, transmit information and/or content (e.g., data, images) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A computer-implemented method for brain mapping and target identification for interventional planning using magnetic resonance imaging (MRI), the method comprising:
receiving, by a computing system that includes at least one processor in communication with at least one memory system and that is in communication to receive data acquired using an MRI system, MR data from the MRI system;
analyzing, by the computer system, the received MR data to monitor and identify motion in real-time;
determining, by the computing system, a set of useable MR data from the acquired MR data based on the identified motion;
generating, by the computer system, a map of the subject's brain based on the set of useable MR data;
identifying, by the computing system, a target location in the subcallosal cingulate (SCC) region of the subject's brain based on the map of the subject's brain, wherein the target location is a point of convergence of multiple fiber bundles passing through the SCC region; and
generating, by the computing system, a report indicating the target location.

2. The computer-implemented method according to claim 1, wherein the multiple fiber bundles passing through the SCC region comprises cingulum bundle (CM), forceps minor (FM), frontal striatal fibers (F-ST), and uncinate fasciculus (UF).

3. The computer-implemented method according to claim 1, further comprising displaying the report on a display.

4. The computer-implemented method according to claim 1, wherein the received MR data is diffusion MR data.

5. The computer implemented method according to claim 4, wherein the received diffusion MR data is acquired using one of diffusion tensor imaging (DTI) or diffusion weighted imaging (DWI).

6. The computer implemented method according to claim 4, wherein the received MR data is acquired for a first number of diffusion directions.

7. The computer-implemented method according to claim 6, further comprising determining additional different directions different from the first number of diffusion directions based on the identified motion and set of useable MR data.

8. The computer implemented method according to claim 7, further comprising receiving, by the computer system, additional MR data acquired for the additional diffusion directions from the MRI system.

9. A system for brain mapping and target identification for interventional planning using magnetic resonance imaging (MRI), the system comprising:
a computing device including a processor programmed to:
receive MR data acquired using an MRI system;
analyze the received MR data to monitor and identify motion in real-time;
determine a set of useable MR data from the acquired MR data based on the identified motion;
generate a map of the subject's brain based on the set of useable MR data;
identify a target location in the subcallosal cingulate (SCC) region of the subject's brain based on the map of the subject's brain, wherein the target location is a point of convergence of multiple fiber bundles passing through the SCC region; and
generate a report indicating the target location; and
a display coupled to the computing device and configured to display the report.

10. The system according to claim 9, wherein the multiple fiber bundles passing through the SCC region comprises cingulum bundle (CM), forceps minor (FM), frontal striatal fibers (F-ST), and uncinate fasciculus (UF).

11. The system according to claim 9, wherein the received MR data is diffusion MR data.

12. The system according to claim 11, wherein the received diffusion MR data is acquired using one of diffusion tensor imaging (DTI) or diffusion weighted imaging (DWI).

13. The system according to claim 11, wherein the received MR data is acquired for a first number of diffusion directions.

14. The system according to claim 13, wherein the processor is further programmed to determine additional diffusion directions different from the first number of diffusion directions based on the identified motion and set of useable MR data.

15. The system according to claim 14, wherein the processor is further programmed to receive additional MR data acquired for the additional diffusion directions from the MRI system.
